(19) United States
(12) Patent Application Publication
(10) Pub. No.: US 2026/0191525 A1
(43) Pub. Date: Jul. 9, 2026

(54) NEEDLE GRASPING AND MANIPULATING DEVICE AND METHOD

(71) Applicant: Thomas Jefferson University, Philadelphia, PA (US)

(72) Inventor: Jeffrey Joseph Valenti, Wappingers Falls, NY (US)

(21) Appl. No.: 19/383,075

(22) Filed: Nov. 7, 2025

Related U.S. Application Data
(63) Continuation of application No. 17/783,320, filed on Dec. 14, 2020, now abandoned.
(60) Provisional application No. 62/947,599, filed on Dec. 13, 2019.

(51) Int. Cl. A61B 17/04; A61B 17/28

(52) U.S. Cl. CPC A61B 17/0483; A61B 17/282; A61B 17/2841

(57) ABSTRACT

A needle holder is described, comprising first and second handles. Each handle comprises a finger loop at a proximal end of the handle, an arm fixedly attached to the finger loop, extending toward a distal end of the handle and defining a longitudinal axis, and a jaw fixedly attached to the arm, extending along the longitudinal axis toward the distal end of the handle. The needle holder further includes a sliding pivot joint connecting the first and second handles at a connection point between the arms and the jaws, the sliding pivot joint configured to allow the first and second handles to move rotationally with respect to one another about a rotational axis orthogonal to the longitudinal axis at the connection point, and/or translationally with respect to one another along the longitudinal axis. A method of grasping a needle is also described.

### CROSS-REFERENCE TO RELATED APPLICATIONS

This application is a continuation of U.S. patent application Ser. No. 17/783,320, filed on Jun. 8, 2022, now pending, which is a 35 U.S.C. § 371 national phase application from, and claiming priority to, International Application No. PCT/US 2020/64747, filed Dec. 14, 2020, which claims priority to U.S. Provisional Ser. No. 62/947,599 , filed on Dec. 13, 2019, all of which are incorporated herein by reference in their entirety.

### FIELD OF THE INVENTION

The present disclosure relates generally to a needle grasping and manipulating device, for example holding a suture needle for suturing tissue at a surgical site.

### BACKGROUND OF THE INVENTION

It is common surgical practice for a physician to join various tissues by passing a needle with attached suture through the tissue. The suture is then tied to hold the tissues in place. Plier-like instruments for gripping and holding suture needles are known in the art - one existing instrument for passing a needle through tissue is a needle holder which usually has a pair of movable, opposed jaws connected to a pair of handles. The handles in turn have a scissor configuration with a locking ratchet mechanism to maintain gripping pressure on the needle held in the jaws of the needle holder. The arms are designed to permit relative pivotal movement between a first closed jaw position wherein the jaw assemblies or units are adjacent to one another and a second open position wherein the jaw assemblies or units are spaced from one another far enough to permit a needle to be inserted therebetween. The jaws are adapted to grasp and hold securely a curved suture needle, whereby pivotal movement of the jaw while in the first closed position causes the suture needle to swing in a corresponding direction for a stitching operation.

Needle holder jaws commonly have a tungsten carbide, serrated surface in a diamond or cross-hatched pattern to enhance the firmness with which the needle is grasped to prevent shifting of the needle while in use. The ratchet mechanism between the scissor handles is locked as the handles close, thereby maintaining firm gripping pressure on the suture needle. Despite this construction, needles are frequently subject to twisting or slipping in the jaws of needle holders as they pass through tissue. Normally the surgeon releases the ratchet mechanism only after the needle has safely passed through the tissue. If the needle is not placed correctly when given to the surgeon or if the needle moves while being used, the surgeon has to stop suturing to take the needle out of the needle holder for the needle to be repositioned. This is time consuming and also puts the surgeon and the operating room staff at risk of a needle stick injury.

Thus, there is a need in the art for a surgical needle holder that can easily adjust the position of a needle both before and as it passes through tissue, in order to minimize time spent intra-operatively adjusting a suture needle and to reduce risk of injury to surgical professionals. The present invention satisfies this need.

### SUMMARY OF THE INVENTION

In one aspect, a needle holder comprises first and second handles, each handle comprising a finger loop at a proximal end of the handle, an arm fixedly attached to the finger loop, extending toward a distal end of the handle and defining a longitudinal axis, and a jaw fixedly attached to the arm, extending along the longitudinal axis toward the distal end of the handle, and a sliding pivot joint connecting the first and second handles at a connection point between the arms and the jaws, the sliding pivot joint configured to allow the first and second handles to move rotationally with respect to one another about a rotational axis orthogonal to the longitudinal axis at the connection point, and/or translationally with respect to one another along the longitudinal axis. In one embodiment, each handle further comprises a slot positioned between the arm and the jaw parallel to the longitudinal axis. In one embodiment, the needle holder further comprises a pin positioned in the slots and joining the two handles together at the connection point.

In one embodiment, the pin comprises two halves removably connected to one another. In one embodiment, the first and second handles each further comprise a ratcheting mechanism directly connected to the finger loops, the ratcheting mechanisms each comprising a set of grooves, the grooves in the first handle configured to mate with the grooves of the second handle. In one embodiment, the grooves are parallel to the longitudinal axis. In one embodiment, each ratcheting mechanism further comprises a second set of grooves perpendicular to the first set of grooves. In one embodiment, the needle holder comprises a material selected from the group consisting of titanium, carbon steel, aluminum, stainless steel, and tungsten carbide. In one embodiment, each jaw comprises a frictional surface comprising a set of gripping elements, the gripping elements directly contacting one another when the jaws are closed.

In one embodiment, the set of gripping elements comprise ridges having a shape selected from the group consisting of triangular, square, rounded, or eccentric. In one embodiment, the set of gripping elements comprises a material selected from the group consisting of titanium, carbon steel, aluminum, stainless steel, and tungsten carbide. In one embodiment, the set of gripping elements directly contact one another when the jaws are closed, but do not interfere with a sliding motion of the needle holder when a needle is positioned between the gripping elements.

In another aspect, a method of grasping a needle comprises the steps of positioning a needle between jaws of a needle grasping device having two handles, closing the jaws of the grasping device around the needle by rotating the two handles about a rotational axis at a connection point, thereby gripping the needle between the jaws, and moving one of the jaws along a longitudinal axis, thereby rotating the needle.

In one embodiment, the jaw is moved by moving one of the handles along the longitudinal axis. In one embodiment, the handle is moved along the longitudinal axis by sliding the handle along a pin positioned in a slot at the connection point. In one embodiment, the method further comprises locking the two handles into a fixed rotational position about the rotational axis by locking a ratcheting mechanism positioned on each handle. In one embodiment, the method further comprises sliding one of the jaws along the longitudinal axis while the two handles are locked in the fixed rotational position about the rotational axis. In one embodiment, the method further comprises releasing the two handles from the fixed rotational position by disengaging the ratcheting mechanism.

In one embodiment, the method further comprises the locking the two handles into a fixed translational position with respect to one another along the longitudinal axis by locking a ratcheting mechanism on each handle. In one embodiment, the method further comprises releasing the two handles from the fixed translational position by disengaging the ratcheting mechanism. In one embodiment, the method further comprises inserting the needle into a tissue of a subject. In one embodiment, the method further comprises the step of releasing the needle from the needle grasping device and re-engaging the grasping device to the needle at another position on the needle.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 is a view of a needle grasping device;

FIG. 2 is a view of a needle grasping device, demonstrating motion of the needle holder when engaging the needle rotation mechanism in one of the two rotating motions;

FIG. 3 is a view of a needle grasping device, demonstrating motion of the needle holder when engaging the needle rotation mechanism in the other of the two rotating motions;

FIG. 4 is a view of a disassembled needle grasping device;

FIG. 5 is a detail view of a connection point of a needle grasping device;

FIG. 6 is a detail view of a finger loop of a needle grasping device with ratchet;

FIG. 7 is a detail view of a connecting pin of a needle grasping device;

FIG. 8 is a detail view of a handle slot of a needle grasping device;

FIG. 9 is an annotated view of a needle grasping device;

FIG. 10 is a view of a disassembled needle grasping device;

FIG. 11 is a detail view of a connecting screw of a needle grasping device;

FIG. 12 is a detail view of a connection point of a needle grasping device;

FIG. 13 is a view of the three pieces of a disassembled needle grasping device; and

FIG. 14 is a method of using a needle grasping device as disclosed herein.

### DETAILED DESCRIPTION

It is to be understood that the figures and descriptions of the present invention have been simplified to illustrate elements that are relevant for a clear understanding of the present invention, while eliminating, for the purpose of clarity, many other elements found in related systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

One aspect of the present invention relates to a needle holder with finger-loop handles, having a ratchet locking mechanism connected to two scissoring arms by the fulcrum that in turn connect to opposing jaws that hold the suture needle. In one embodiment, the needle holder has an overlapping fulcrum and elongated ratchet locking mechanism, and an additional locking mechanism in the grooves of a traditional ratchet design. In some embodiments a needle holder includes a sliding mechanism, configured to manipulate and move needles along any plane parallel to the longitudinal axis of the device. The sliding mechanism enables increased movement, which means the needle can be rotated up to 360 degrees while in the jaws. Certain embodiments include a second locking mechanism that will secure the needle holder sliding position in place once the needle has been moved to the desired position.

The disclosed design allows quick placement and adjustment of the needle in the holder and maintains proper, secure alignment of the needle to the jaws of the needle holder. This provides the surgeon with an implement to facilitate driving a needle suture in difficult conditions of long reaching tissues or limited space, such as when reaching down into body cavities or intra-orally, and to adjust the direction of the needle while actively suturing. This will make it easier for surgeons to handle and manipulate the instrument in relatively small and sometimes minute increments and degrees while suturing and not have to readjust the needle with the surgeon's hand to get better angulation for placement of a suture. The sliding system configuration in the needle holder adds more control of a suturing operation by increasing comfort.

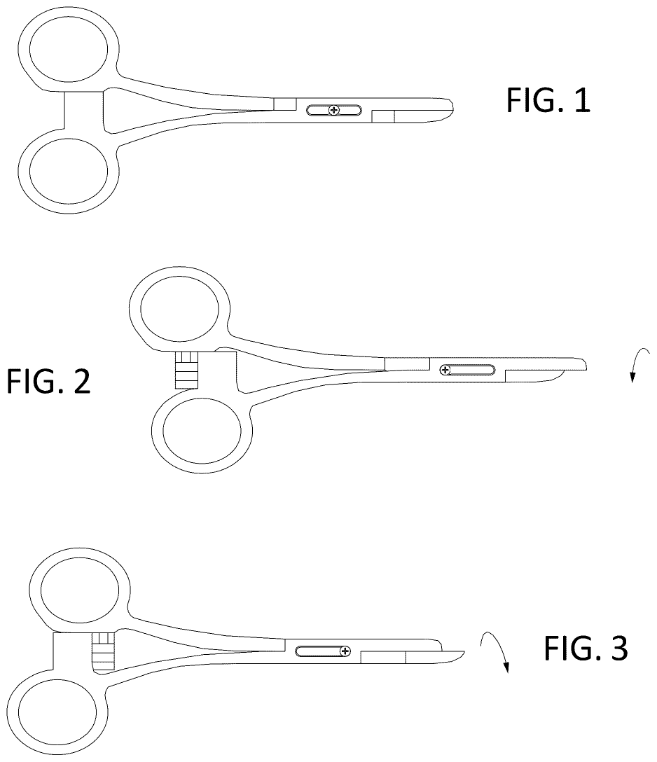

Referring now to FIGS. 1, 2, and 3, a needle holding instrument of the present invention is shown, with the embodiment of a specialized overlapping fulcrum and an elongated ratchet locking mechanism to aid in the sliding mechanism of the needle holder. FIG. 1 shows the position of the corresponding segments when the two handles are aligned. FIG. 2 shows the position of the corresponding segments when the bottom handle is pushed toward the jaws. Moving the bottom handle toward the jaws as shown has the effect of turning a needle grasped in the jaws in a clockwise direction as shown. FIG. 3 shows the position of the corresponding segments when the top handle is pushed toward the jaws, which has the effect of turning a needle grasped in the jaws in a counterclockwise direction as shown. In some embodiments, the needle holder is constructed of surgical stainless steel, carbon steel, titanium, or aluminum, but other materials can also be used. In some embodiments, all components of the needle holder are constructed from the same material, while in other embodiments, some components may be constructed from a different material than others. In some embodiments, a needle holder may include tungsten carbide inserts in the tips that grasp the needle. Tungsten carbide inserts are more durable than stainless steel, last longer, and typically offer a better grip.

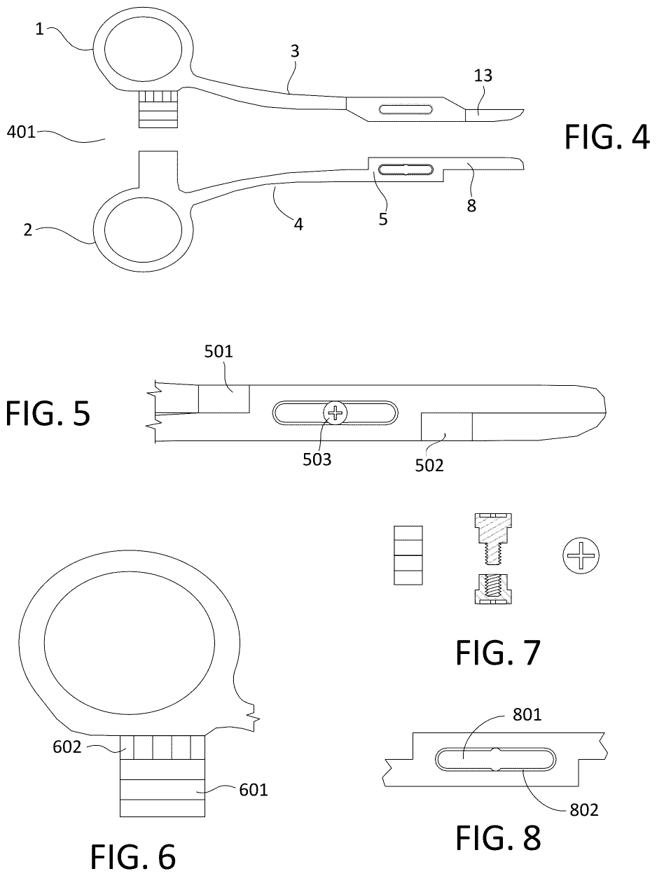

With reference to FIG. 4, in the depicted embodiment, at the proximal end 401 finger loops 1 and 2 are of a size to accept thumb or finger within the loop. The finger loops are in turn connected to arms 3 and 4, fulcrum 5, and jaws 8 and 13. Finger loop 1, arm 3, and jaw 13 form a first handle of the device, while finger loop 2, arm 4, and jaw 8 form a second handle of the device. The user can push with their thumb or fingers to cause one of the handles to slide forward or back in relation to the opposing handle, which will in turn cause a needle grasped in the jaws to rotate, thereby changing the angle and/or direction of the needle.

FIG. 6 shows a detail view of finger loop 1 and attached two-dimensional ratchet mechanism that can easily be selectively used by a surgeon that will secure the needle holder sliding position in place once the needle has been moved to the desired position. The two-dimensional ratchet lock consists of two component ratchet mechanisms, the first mechanism comprising a set of horizontal (i.e. parallel to the direction of the handle) notches 601, and the second mechanism comprising a set of vertical (i.e. perpendicular to the direction of the handle) notches 602. The sliding ratchet mechanism may provide no notches, 1 notch, 2 notches, 3 notches, 4 notches, or the like. In some embodiments, the first mechanism has the same number of notches as the second mechanism, although in other embodiments they may be different.

The first handle and the second handle have matching notches on their opposing surfaces which engage one another, locking, as the finger loops are brought together. These notches are longitudinal to the needle holder. Such locking ratchet mechanisms are known in the art, but the elongated design allows for the needle holder to remain locked around the needle when a sliding maneuver is performed. While in some embodiments the finger loops are substantially round, in other embodiments the finger loops may have other shapes, for example rectangular, triangular, or a castrovie.

Another advantage of the disclosed design is that if the needle holder is closed tighter to engage the more proximal notches, it is configured to provide higher resistance when engaging the sliding mechanism to allow the surgeon to have more preference for the level of resistance when engaging the sliding mechanism. The vertical notches of the ratchet 602 are configured to prevent the handles from sliding relative to one another when the surgeon does not desire this additional function.

As shown in FIG. 4, the arms 3 and 4 are of equal length and terminate at a sliding pivot joint 5. As shown in FIG. 5, the arms have a step off ledges 501 and 502 with extra length that allows either arm to slide slightly under the pivot joint 503. The area when the arms meet the sliding pivot joint is sloped at the area of the pivot joint so as to prevent the suture material from being caught or tangled at the junction of the two parts. There is also a sloped area where the pivot joint meets the jaws at the area of the jaws to prevent the suture material from being caught at the junction of the two parts. The pivot joint is constructed such that the arms of the needle holder terminate in an elongated flat surface. The flat surface of one arm passes through a matched opening in the other arm with both being united to one another by a pin (see FIG. 7) passing through the center of the flat zone. Both arms pivot about this pin establishing the scissors action of the needle holder. The pin in the disclosed device is on a track which allows for movement of either corresponding handle of the needle holder down the pin's longitudinal track (see FIG. 8) which causes a sliding movement of the instrument. This configuration allows the jaws either to be aligned (as shown in FIG. 1) or to slightly offset their position in relation to each other (as shown in FIG. 2 and FIG. 3) which will cause a rotation of the needle when it is held in the jaws which causes changing its position to facilitate better position for suture placement.

As shown in FIG. 5, in one embodiment the jaws are substantially straight, while in other embodiments, the jaws may be curved or otherwise shaped depending on requirements. The opposing jaws at the end of each handle may comprise one or more ridges, which may have a triangular, square, rounded, eccentric, or other shape.

In one embodiment, the track has a length of between 5 mm and 20 mm, and can vary based on the overall size of the needle holder and location it is being used on the body. This is an average track length, but in some embodiments a device can have a track length as small as one millimeter, for example in embodiments that are used for very delicate procedures such as using needle holders for anastomosis of small arteries or nerves. As shown in FIG. 7, in some embodiments the pin comprises two interlocking parts, which may connect for example via interlocking threads, a snapping mechanism, or the like. In one embodiment, the pin may be built in or removably attached in a fixed position to one of the handles, and may be constructed with an oblong shape that is removable from the slot on the other handle when in one orientation (for example rotated 90 degrees from the working orientation shown in FIGS. 1-3), while slidably locked into the slot when the arms in the working orientation shown in FIGS. 1-3.

FIG. 7 shows a detail view of a screw used to connect the two handles and engage the sliding mechanism. FIG. 8 shows a detail view of the slot, showing a slight groove 802 in the middle position along the slot 801 which allows for easier repositioning of the two handles to an aligned position. In some embodiments, the slot with a slight groove may be absent.

The finger loops 1 and 2 and arms 3 and 4 allow the surgeon to grasp the instrument and apply pressure to close it. Once closed, the ratchet mechanism locks to maintain the closed position while still allowing the two handles to slide in relation to each other. The ratchet mechanism can be further closed to engage a second locking mechanism to prevent the segments from sliding. The ratchet mechanism is opened by the surgeon applying opposing pressure to the finger loops. The pivot joint allows for ease of opening and closing the jaws. The needle holder jaws taper in both width and height to a terminal, rounded point. This pointed tip allows for good directional placement and visualization of needle position by the surgeon using the instrument. When the handles are slid relative to one another, the jaws will sometimes not be lined up depending on the desired rotation of the needle at any given time.

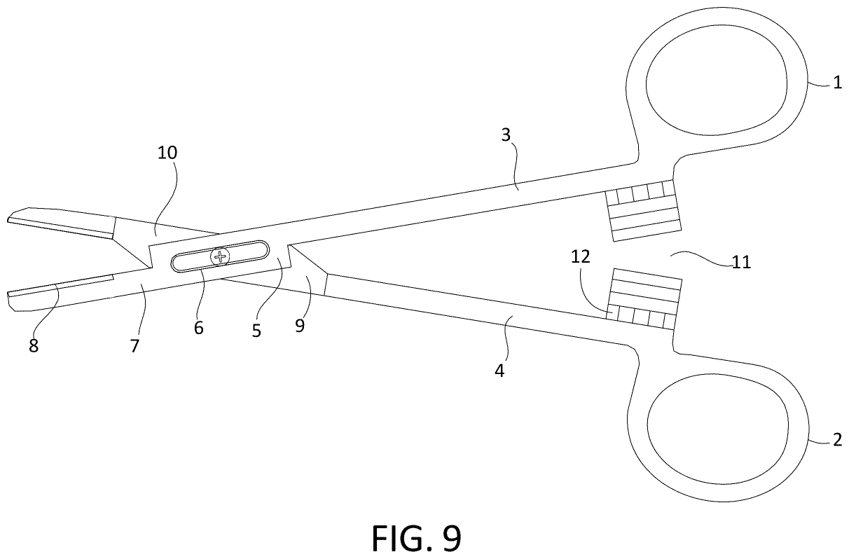

With regard to FIG. 9, an annotated view of a needle grasping device is shown. The exemplary device includes finger loops 1 and 2, ratcheting elements 11 and 12, arms 3 and 4, connection point 5 including pin 6, grooves 9 and 10, jaw 7 and jaw frictional gripping surface 8.

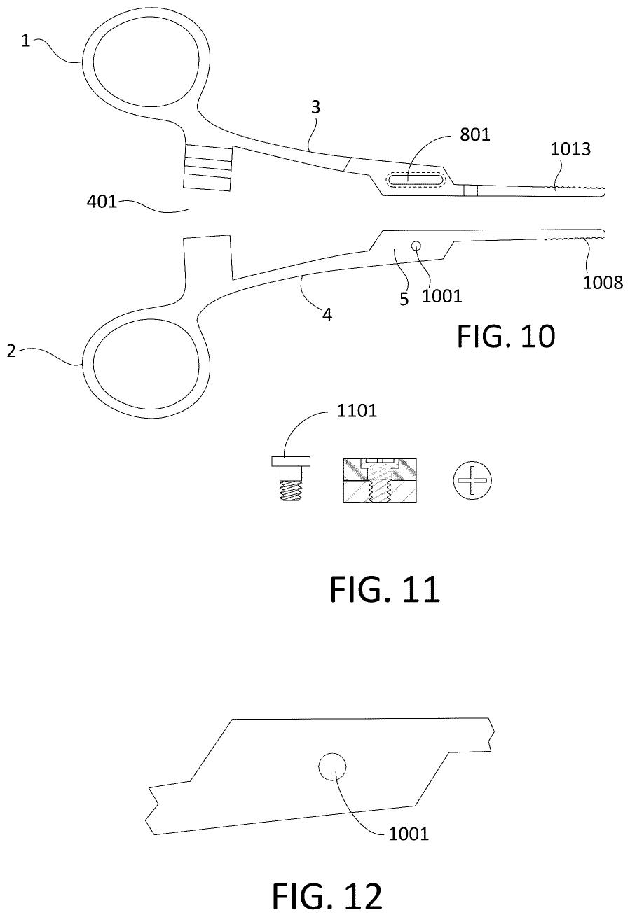

With reference to FIG. 10, FIG. 11, and FIG. 12, another embodiment of a needle grasping device is shown. With reference to FIG. 10, in the depicted embodiment, arms 3 and 4 include slightly curved portions connecting finger loops 1 and 2 to their respective connection points 5. Additionally, in the depicted embodiment, connection point 5 includes slot 801 on arm 3, and hole 1001 on arm 4. Hole 1001 may in some embodiments be threaded. A cross-sectional view of the connection point and detail view of the attaching screw is shown in FIG. 11. In the depicted embodiment, hole 1001 is threaded and screw 1101 engages the threads. In the depicted embodiment, screw 1101 is partially threaded along the bottom portion to engage the threads in hole 1001, while the top portion of the screw is not threaded so as to reduce friction with slot 801. In one embodiment, hole 1001 is not threaded, and a rivet may be used in place of the attaching screw. The head of screw 1101 is sufficiently wide such that while the threaded portion of screw 1101 fits through slot 801, the head does not. The screw head may thus hold arm 3 against arm 4 at connection point 5, while still allowing arm 3 to slide with respect to arm 4 along slot 801. A detail view of hole 1001 is shown in FIG. 12. As shown in FIG. 10 of the depicted embodiment, the jaws 1013 extend further from connection point 5 than in the embodiment of FIG. 9, and each jaw includes frictional gripping surface 1008.

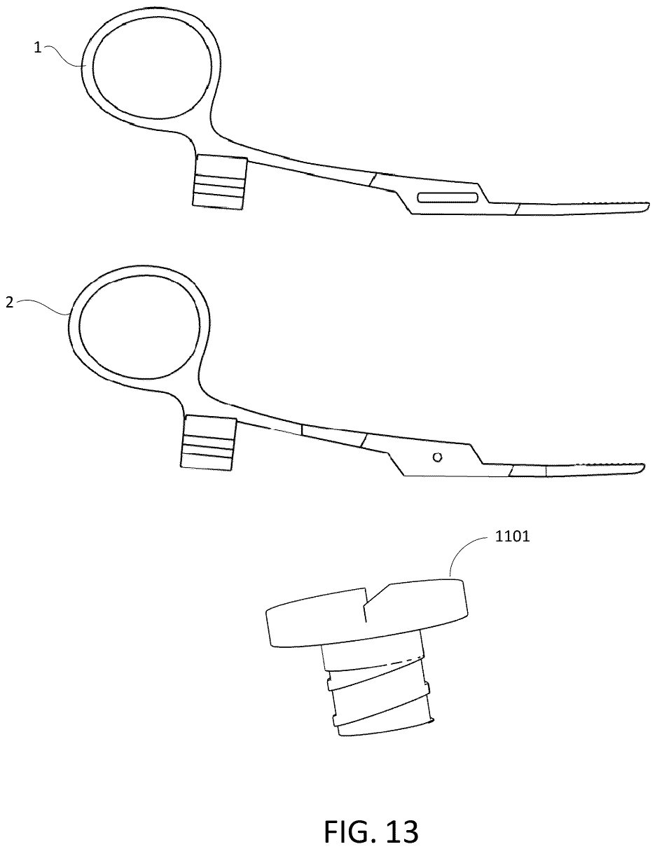

With reference to FIG. 13, an alternate embodiment of arms 1 and 2 and screw 1101 is shown.

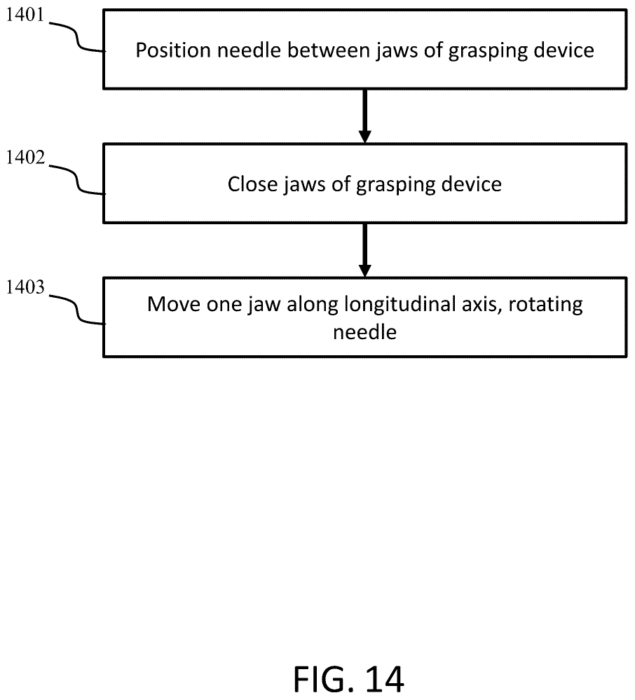

In another aspect, the present disclosure includes methods of grasping and/or positioning a needle, for example using a device as disclosed herein. With reference now to FIG. 14, an exemplary method includes steps of positioning a needle between jaws of a needle grasping device in step 1401, closing the jaws of the grasping device around the needle thereby gripping the needle between the two jaws of the grasping device in step 1402, and moving one jaw of the grasping device along a longitudinal axis, thereby causing the needle to rotate in step 1403. The jaw may be moved by various methods, for example in one embodiment the jaw is moved by moving one handle of the grasping device to which the jaw is fixedly attached, the handle being slidably connected to an opposing handle, the opposing handle itself fixedly connected to the opposing jaw.

In some embodiments, the method may further comprise the step of locking two handles of a needle grasping device into a fixed position relative to one another along one or two axes. In one embodiment, each handle of the needle comprises a ratcheting mechanism having grooves extending in the longitudinal direction, the grooves on each handle configured to mate with one another when the grasping device is in a closed position. A method may comprise the step of closing the grasping device and mating the grooves in the respective handles, thereby holding the grasping device in substantially the same closed position when the handles are released. In such embodiments, a method may further comprise a step of sliding the two handles back and forth relative to one another along the longitudinal axis while the handles are locked together, along the longitudinal grooves. In some embodiments, the ratcheting mechanism further comprises a set of grooves perpendicular to the longitudinal grooves on each handle, and configured to further hold the handles in a fixed position along the longitudinal axis, for example when the two handles are slid along the longitudinal axis into a new position with respect to one another (and consequently in some embodiments, when a needle grasped between the jaws is rotated). A method of using the grasping device may then further comprise the step of moving the handles into a fixed position along the longitudinal axis and locking the handles in place by engaging the perpendicular grooves in each handle.

Methods related to the disclosed device may further include surgical steps, for example piercing a needle grasped between the handles through one or more tissues, releasing the grasping device from the needle, and re-engaging the grasping device to the needle at a position on the other side of the one or more tissues in order to pull the needle fully through the tissue, for example for placing a suture. In some embodiments, the needle may be rotated before, after, or during the piercing step, using the mechanism described above. In some embodiments, a method may include the step of locking the handles of the grasping device into a fixed position relative to one another, either to keep the opposing jaws at a fixed distance/grasping force with respect to one another, or to keep the handles at a fixed position with respect to one another along the longitudinal axis.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A needle holder, comprising:
first and second handles, each handle comprising:
a finger loop at a proximal end of the handle;
an arm fixedly attached to the finger loop, extending toward a distal end of the handle and defining a longitudinal axis;
a jaw fixedly attached to the arm, extending along the longitudinal axis toward the distal end of the handle; and
a slot positioned between the arm and the jaw parallel to the longitudinal axis; and
a sliding pivot joint positioned in the slots in the first and second handles, connecting the first and second handles at a connection point between the arms and the jaws, the sliding pivot joint configured to allow the first and second handles to move rotationally with respect to one another about a rotational axis orthogonal to the longitudinal axis at the connection point, and/or translationally with respect to one another along the longitudinal axis.

2. The needle holder of claim 1, wherein the pin comprises two halves removably connected to one another.

3. The needle holder of claim 1, wherein the first and second handles each further comprise a ratcheting mechanism directly connected to the finger loops, the ratcheting mechanisms each comprising a set of grooves, the grooves in the first handle configured to mate with the grooves of the second handle.

4. The needle holder of claim 3, wherein the grooves are parallel to the longitudinal axis.

5. The needle holder of claim 4, wherein each ratcheting mechanism further comprises a second set of grooves perpendicular to the first set of grooves.

6. The needle holder of claim 1, wherein the needle holder comprises a material selected from the group consisting of titanium, carbon steel, aluminum, stainless steel, and tungsten carbide.

7. The needle holder of claim 1, wherein each jaw comprises a frictional surface comprising a set of gripping elements, the set of gripping elements directly contacting one another when the jaws are closed.

8. The needle holder of claim 7, wherein the set of gripping elements comprise ridges having a shape selected from the group consisting of triangular, square, rounded, or eccentric.

9. The needle holder of claim 8, wherein the set of gripping elements comprises a material selected from the group consisting of titanium, carbon steel, aluminum, stainless steel, and tungsten carbide.

10. The needle holder of claim 9, wherein the set of gripping elements directly contact one another when the jaws are closed, but do not interfere with a sliding motion of the needle holder when a needle is positioned between the set of gripping elements.

11. The needle holder of claim 1, wherein each of the first and second handles further comprises a sloped feature near the pivot joint configured to prevent a material held by the needle holder from becoming caught between the handles near the pivot joint.

12. The needle holder of claim 1, wherein each of the first and second handles comprises an elongated flat surface between the arm and the jaw configured to be in direct contact with one another.

13. A method of grasping a needle, comprising the steps of:
positioning a needle between jaws of a needle grasping device having two handles;
closing the jaws of the grasping device around the needle by rotating the two handles about a rotational axis at a connection point, thereby gripping the needle between the jaws; and
moving one of the jaws along a longitudinal axis, thereby rotating the needle.

14. The method of claim 13, wherein the jaw is moved by moving one of the handles along the longitudinal axis.

15. The method of claim 14, wherein the handle is moved along the longitudinal axis by sliding the handle along a pin positioned in a slot at the connection point.

16. The method of claim 13, further comprising locking the two handles into a fixed rotational position about the rotational axis by locking a ratcheting mechanism positioned on each handle.

17. The method of claim 16, further comprising sliding one of the jaws along the longitudinal axis while the two handles are locked in the fixed rotational position about the rotational axis.

18. The method of claim 16, further comprising releasing the two handles from the fixed rotational position by disengaging the ratcheting mechanism.

19. The method of claim 13, further comprising the locking the two handles into a fixed translational position with respect to one another along the longitudinal axis by locking a ratcheting mechanism on each handle.

20. The method of claim 19, further comprising releasing the two handles from the fixed translational position by disengaging the ratcheting mechanism.

21. The method of claim 13, further comprising inserting the needle into a tissue of a subject.

22. The method of claim 21, further comprising the step of releasing the needle from the needle grasping device and re-engaging the grasping device to the needle at another position on the needle.
